(19) United States
(12) Patent Application Publication
(10) Pub. No.: US 2007/0207545 A1
(43) Pub. Date: Sep. 6, 2007

(54) Novel animal cell expression vectors

(75) Inventors: Masahiro Asada, Ibaraki (JP); Toru Imamura, Ibaraki (JP); Emi Honda, Ibaraki (JP)

Correspondence Address: SUGHRUE MION, PLLC, 2100 PENNSYLVANIA AVENUE, N.W., SUITE 800, WASHINGTON, DC, 20037, US

(73) Assignee: NATIONAL INSTITUTE OF ADVANCED INDUSTRIAL SCIENCE AND TECHNOLOGY

(21) Appl. No.: 11/603,050

(22) Filed: Nov. 22, 2006

(30) Foreign Application Priority Data
Feb. 2, 2006 (JP) 2006-026215

(51) Int. Cl. C12N 15/09; C07H 21/04

(52) U.S. Cl. 435/455; 435/320.1; 536/23.2

(57) ABSTRACT

In the present invention, in order to expand the range of drug resistance markers available for use in a transgenic vector for animal cells, an expression vector comprising a multicloning site identical to those of the existing vectors and drug resistance marker genes different from those of the existing vectors is produced. The present invention provides an animal cell expression vector containing a multicloning site and a puromycin resistance gene or a blasticidin S resistance gene as a drug resistance marker gene, the expression of which is controlled by SV40 ori and SV40 pA.

### CROSS-REFERENCE TO RELATED APPLICATION

This application claims priority of Japanese patent application No. 2006-026215 filed on Feb. 2, 2006.

### BACKGROUND OF THE INVENTION

1. Field of the Invention

The present invention relates to an expression vector that is used to introduce genes into cells, particularly animal cells.

2. Description of Related Art

There are a number of existing expression vectors that are used to introduce genes into mammalian cells. Such vectors each generally comprise: 1) a multicloning site (MCS) into which an insert DNA is cloned; 2) a promoter and a poly(A) addition sequence necessary for the expression of the insert; 3) a drug resistance marker gene which allows selection of the gene-transfected cells, as well as a promoter and a poly(A) addition sequence necessary for the expression of the drug resistance marker gene; 4) an ori sequence which allows replication of the vector in a prokaryotic cell; 5) an antibiotic resistance gene which allows drug selection of the gene-transfected prokaryotic cells; and/or the like.

When more than one foreign gene is transferred into a single cell, in general, different vectors having different drug resistance marker genes are used. Thus, it is necessary to prepare insert DNA fragments containing each foreign gene which are compatible with restriction sites in the MCS of vectors to be used.

Also, upon transfection of more than one foreign gene, varied combinations of the inserts and the drug resistance marker genes are often used. However, there are few existing vectors that have the same MCS but have different drug resistance marker genes. Thus, when transfection of multiple genes are carried out using vectors having different drug resistance marker genes, it frequently takes a lot of work to clone inserts into MCSs having different nucleotide sequences in the vectors by, for example, adding a linker to the inserts or newly preparing the inserts in forms compatible with each vector.

pcDNA3.1, pcDNA3.1/Hygro and pcDNA3.1/Zeo are a few examples of the vectors that have the same MCS but have different drug resistance marker genes. pcDNA3.1/Hygro and pcDNA3.1/Zeo both comprise the same MCS as pcDNA3.1 which comprises a neomycin resistance gene, and further comprise a hygromycin resistance gene and a zeocin resistance gene, respectively. These vectors pcDNA3.1, pcDNA3.1/Hygro, and pcDNA3.1/Zeo share common MCS, and thus, inserts cloned in the vectors can be easily replaced each other in order to change the combinations of the drug resistance marker genes and the inserts. However, procedures for producing pcDNA3.1/Hygro and pcDNA3.1/Zeo have not been published.

Meanwhile, recently, in addition to neomycin, hygromycin, and zeocin, antibiotics that are more inexpensive and can be used for selection of transgenic cells with higher efficiencies have been developed. Examples thereof include puromycin and blasticidin S. However, the existing expression vectors that can provide the resistances to such antibiotics have completely different vector backgrounds, in particular, completely different MCS sequences, from those of pcDNA3.1 and the like. Therefore, in order to transfect cells with a foreign gene which was once cloned into pcDNA3.1 such that the transfected cells can be selected in the presence of puromycin or blasticidin S, it is necessary to newly insert the foreign gene into the vectors containing the puromycin resistance gene or the blasticidin S resistance gene. The insertion process further requires a step of preparing the insert so as to have both terminal sequences that are compatible with the MCS of the vectors containing the puromycin resistance gene or the blasticidin S resistance gene.

### SUMMARY OF THE INVENTION

It is an objective of the present invention to expand the range of drug resistance markers available for use in the expression vectors having a multicloning site (MCS) identical to those of the vectors pcDNA3.1, pcDNA3.1/Hygro, and pcDNA3.1/Zeo and thereby to provide improved convenience for use of pcDNA3.1-based animal cell expression vectors. For this purpose, the present invention relates to the construction of a new expression vector comprising a multicloning site (MCS) identical to those of the vectors pcDNA3.1, pcDNA3.1/Hygro, and pcDNA3.1/Zeo and having puromycin or blasticidin S resistance as a drug resistance marker, instead of the neomycin resistance.

In order to attain the above objective, we first considered a way to remove a neomycin resistance gene from pcDNA3.1 and then insert a puromycin resistance gene or a blasticidin S resistance gene thereinto.

However, there is no restriction enzyme sites that sandwich the neomycin resistance gene and are useful for removing the neomycin resistance gene alone from pcDNA3.1 without destroying a promoter region (SV40 ori) that regulates the expression of the gene and a poly(A) addition sequence (SV40 pA). Thus, we focused on a unique SmaI recognition site that exists between the neomycin resistance gene and SV40 ori, which functions as a promoter of the neomycin resistance gene contained in a pcDNA3.1 vector. Also, we focused on an NaeI recognition site that exists downstream of the neomycin resistance gene and upstream of the SV40 poly(A) addition sequence (hereinafter also referred to as “SV40 pA”). As a result of intensive studies in view of these sites, we first succeeded in substituting the neomycin resistance gene, the expression of which is controlled by the above SV40 ori and SV40 pA, with a puromycin resistance gene or a blasticidin S resistance gene, in the pcDNA3.1. This has led to the completion of the present invention.

That is, the present invention encompasses the following (1) to (8):

(1) an animal cell expression vector comprising a multicloning site and a drug resistance marker gene, the expression of which is controlled by SV40 ori and SV40 pA;

wherein the drug resistance marker gene is a puromycin resistance gene or blasticidin S resistance gene;

(2) the animal cell expression vector described in the above (1), wherein the multicloning site contains restriction enzyme recognition sites that are identical to those contained in a multicloning site of either pcDNA3.1(+) or pcDNA3.1(−), and the restriction enzyme recognition sites are located in the same order as those of pcDNA3.1(+) or pcDNA3.1(−);

(3) a DNA fragment comprising a puromycin resistance gene in which SmaI recognition sites are added to both 3′ and 5′ ends of the puromycin resistance gene and an NaeI recognition site is added upstream of the SmaI recognition site on the 3′ end;

(4) the DNA fragment described in the above (3), comprising the nucleotide sequence as set forth in SEQ ID NO: 4;

(5) a DNA fragment comprising a blasticidin S resistance gene in which SmaI recognition sites are added to both 3′ and 5′ ends of the blasticidin S resistance gene and an NaeI recognition site is added upstream to the SmaI recognition site on the 3′ end;

(6) the DNA fragment described in the above (5), comprising the nucleotide sequence as set forth in SEQ ID NO: 3;

(7) a plasmid vector comprising the nucleotide sequence as set forth in SEQ ID NO: 1; and

(8) a plasmid vector comprising the nucleotide sequence as set forth in SEQ ID NO: 2.

In accordance with the present invention, a drug resistance gene the expression of which is controlled by SV40 ori and SV40 pA has been substituted with a puromycin resistance gene or blasticidin S resistance gene in an animal cell expression vector containing a multicloning site (MCS). Thus, upon selection of recombinant cells containing a foreign gene, a wider range of available drug selection markers is provided so that gene introduction can be carried out with improved efficiency.

Also, in particular, the vectors pcDNA3.1(+)-PUR and pcDNA3.1(+)-BSD of the present invention, which are obtained by substituting the neomycin resistance gene within pcDNA3.1(+) with a puromycin resistance gene and with a blasticidin S resistance gene, respectively, have MCSs identical to those of existing vectors such as pcDNA3.1(+), pcDNA3.1/Hygro (+), and pcDNA3.1/Zeo (+). Thus, inserts cloned into the vectors are easily exchangeable one another. Therefore, in accordance with the present invention, change of drug selection markers is significantly simplified and a wider range of such markers becomes available.

### BRIEF DESCRIPTION OF THE DRAWINGS

FIG. 1 schematically shows the outline of a process for constructing a pcDNA3.1(+)-BSD vector.

FIG. 2 shows photographs showing results of the cultures of stably expressing cells obtained using pcDNA3.1(+), pcDNA3.1(+)-PUR, and pcDNA3.1(+)-BSD in the presence of different antibiotics for 6 days.

### DESCRIPTION OF THE PREFERRED EMBODIMENTS

pcDNA3.1(+) (Invitrogen, Carlsbad, Calif., USA) is a specific example of an expression vector used for animal cells that has a multicloning site (MCS) and drug resistance gene the expression of which is controlled by SV40 ori and SV40 pA.

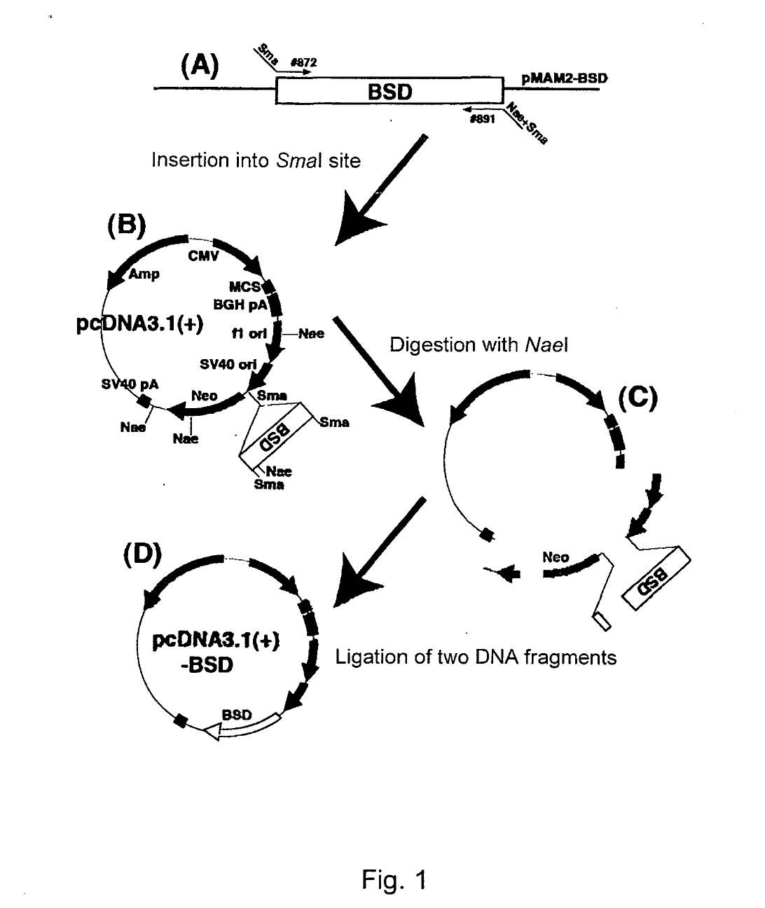

The structure of pcDNA3.1(+) is shown in FIG. 1 (B). pcDNA3.1(+) has a multicloning site (MCS) and an ampicillin resistance gene (Amp) and a neomycin resistance gene (Neo) as drug resistance genes. Among them, the neomycin resistance gene is disposed between SV40 ori that functions as a promoter of the neomycin resistance gene and SV40 poly(A) addition sequence, and the expression of the resistance gene is controlled by SV40 ori and SV40 pA. The multicloning site (MCS) is composed of a plurality of restriction enzyme recognition sites, and the restriction enzyme recognition sites are located in the direction of BGH pA in the following order from the promoter: NheI, PmeI, AflII, HindIII, Asp7181, KpnI, BamHI, BstXI, EcoRI, EcoRV, BstXI, NotI, XhoI, XbaI, DraII, ApaI, and PmeI sites.

Likewise, in the case of pcDNA3.1(−) (Invitrogen, Carlsbad, Calif., USA), the restriction enzyme recognition sites are located in the direction of BGH pA in the following order from the promoter: NheI, PmeI, DraII, ApaI, XbaI, XhoI, NotI, BstXI, EcoRV, EcoRI, BstXI, BamHI, Asp7181, KpnI, HindIII, AflII, and PmeI sites.

Specifically, in the vector of the present invention, the neomycin resistance gene of the above vectors pcDNA3.1(+) or pcDNA3.1(−) has been substituted with a puromycin resistance gene or a blasticidin S resistance gene. Also, the vector of the present invention comprises restriction enzyme recognition sites identical to the above described restriction enzyme recognition sites within the multicloning site of the vectors, wherein the recognition sites are located in the same order as the above restriction enzyme recognition sites.

However, the existence of such identical restriction enzyme recognition sites that are located in an identical order does not mean that the vectors of the present invention have an identical nucleotide sequence to those of pcDNA3.1(+) or pcDNA3.1(−) throughout whole region of their multicloning sites.

Hereafter, the method of constructing the expression vector used for animal cells of the present invention is exemplified with reference to FIG. 1 based on an example that makes use of a blasticidin S resistance gene.

In pcDNA3.1(+), one of NaeI recognition sites exists between the neomycin resistance gene and the SV40 poly(A) addition sequence, and a unique SmaI recognition site exists between the neomycin resistance gene and SV40 ori. Using the SmaI recognition site, it is possible to insert a blasticidin S resistance gene into pcDNA3.1(+) by adding a SmaI recognition site to both ends of the blasticidin S resistance gene, followed by ligation of the blasticidin S resistance gene into SmaI-digested pcDNA3.1 (+) with a Jigase.

However, pcDNA3.1(+) does not contain appropriate restriction enzyme recognition sites that can be used for removing the neomycin resistance gene. Thus, it is difficult to remove the neomycin resistance gene from pcDNA3.1(+) in a simple procedure. In pcDNA3.1(+), one of NaeI recognition sites exists between the neomycin resistance gene and the SV40 poly(A) addition sequence; however, the NaeI is not a unique site and other Nae I recognition site also exists upstream of SV40 ori. Thus, cleavage using Nae I results in removal of SV40 ori so that the obtaining of sufficient drug resistance cannot be expected.

Thus, in accordance with the present invention, DNA fragment containing the blasticidin S resistance gene is prepared in a manner such that a SmaI recognition site is added to the 5′ end of the blasticidin S resistance gene, a SmaI recognition site is added to the 3 end thereof, and an NaeI recognition site is also added upstream of the SmaI recognition site on the 3′ end (FIG. 1 (A)). The nucleotide sequence of the resulting DNA fragment is set forth in SEQ ID NO: 3 in the Sequence Listing. Thereafter, the DNA fragment is prepared into complete double-strand DNA fragment and then ligated into the SmaI-digested pcDNA3.1(+) using ligase (FIG. 1 (B)). Then, the blasticidin S resistance gene-cloned pcDNA3.1(+) is digested with NaeI to produce a DNA fragment containing the blasticidin S resistance gene that has Noel-cleaved ends. Further, at such time, in addition to the DNA fragment containing the blasticidin S resistance gene, DNA fragments derived from the anterior half and the posterior half of the neomycin resistance gene, respectively, and a DNA fragment derived from pcDNA3.1(+) vector backbone are produced (FIG. 1 (C)). These fragments have different molecular weights each other, and thus the DNA fragment containing the blasticidin S resistance gene can be separated by agarose gel electrophoresis.

Subsequently, the thus separated DNA fragment containing the blasticidin S resistance gene having Noel-cleaved ends is ligated into NaeI-cleaved arms derived from the NaeI recognition site that exists downstream of the neomycin resistance gene and upstream of the SV40 poly(A) addition sequence in pcDNA3.1(+), in which the neomycin resistance gene has been removed therefrom. As a result, the vector pcDNA3.1(+)-BSD (SEQ ID NO: 1) in which the neomycin resistance gene has been substituted with the blasticidin S resistance gene is obtained (FIG. 1 (D)).

In addition, for inserting a puromycin resistance gene into the vectors, DNA fragment containing the puromycin resistance-gene is prepared in a manner such that a SmaI recognition site is added to the 5′ end of the puromycin resistance gene, a SmaI recognition site is added to the 3′ end thereof, and an Noel recognition site is also added upstream of the SmaI recognition site on the 3′ end. The nucleotide sequence of the resulting DNA fragment is set forth in SEQ ID NO: 4. Thereafter, in the same manner as described above in the case of the blasticidin S resistance gene, the vector pcDNA3.1(+)-PUR (SEQ ID NO: 2) in which the neomycin resistance gene of pcDNA3.1(+) has been substituted with a puromycin resistance gene is obtained.

The vectors pcDNA3.1(+)-BSD and pcDNA3.1(+)-PUR that have been newly constructed in accordance with the present invention have MCSs completely identical to those of the existing vectors pcDNA3.1(+), pcDNA3.1/Hygro(+), and pcDNA3.1/Zeo(+). Thus, once an insert being compatible with one of those vectors was prepared, it can be directly inserted into any one of these vectors. In addition, an insert that was inserted into one of such vectors can readily be cleaved and inserted into the other vectors. Thus, it becomes easy to insert a single gene into different vectors each containing different drug resistance marker genes. Thus, the vectors of the present invention can be very useful tools for analysis of functions of a plurality of genes.

For instance, in biosynthesis of carbohydrate in animal cells, functional end products, i.e., glycoproteins, are produced via concerted actions of a plurality of gene products (proteins) such as sugar-nucleotide transporters, glycosyltransferase, and glycolytic enzymes. In addition, recently, it has been reported that a plurality of gene products form a complex in a cell such that the function of the complex is exhibited therein. In order to totally understand the function of such complex, it is necessary to introduce a plurality of the genes in a variety of combinations into host cells and verify the effects of such introductions. Thus, a panel of the obtained vectors according to the present invention can be useful tools for such studies.

The vectors of the present invention, pcDNA3.1(+)-BSD comprising the blasticidin S resistance gene (BSD) which comprises the nucleotide sequence as set forth in SEQ ID NO: 1, and pcDNA3.1(+)-PUR comprising the puromycin resistance gene (PUR) which comprises the nucleotide sequence as set forth in SEQ ID NO: 2, were deposited (the deposited samples of which are referred to as pcDNA3.1(+)-BSD/c1.8 and pcDNA3.1(+)-PUR/c1.9, respectively) at the International Patent Organism Depositary of the National Institute of Advanced Industrial Science and Technology (Tsukuba Central 6, 1-1-1 Higashi, Tsukuba, Ibaraki, Japan) as of Dec. 9, 2005 under the accession numbers: FERM P-20732 and FERM P-20733, respectively (the original deposits). Requests for transfer of the depositions from the original to the international deposition under the Budapest Treaty were received at the International Patent Organism Depositary of the National Institute of Advanced Industrial Science and Technology on Oct. 27, 2006 under the receipt numbers: FERM ABP-10716 and FERM ABP-10717, and then accepted as of ______ under the accession numbers: FERM BP-10716 and FERM BP-10717, respectively.

### EXAMPLES

### Example 1

1. Preparation of BSD DNA Fragments

A blasticidin S resistance gene (BSD) was amplified by PCR using a vector pMAM2-BSD (Kaken Pharmaceutical Co., Ltd., Tokyo, Japan) which contains such gene, as a template. As a sense primer, an oligonucleotide comprising a SmaI site to be added at the 5′ end thereof was used. Also, as an antisense primer, an oligonucleotide comprising SmaI plus NaeI sites to be added at the 5′ end thereof was used. Specifically, the sense primer #872 (5′-tcc ccc ggg tca ata tgc ctt tgt ctc a-3′) (SEQ ID NO: 5) and the antisense primer #891 (5′-taa ccc ggg ccg gct ggt gct tag ccc tcc c-3′) (SEQ ID NO: 6) were used to specifically amplify BSD with thermostable polymerase KOD-plus-(FIG. 1 (A)).

The thus obtained DNA fragment was cloned into a pCR-Blunt II-TOPO vector using a Zero Blunt TOPO PCR Cloning Kit. Then, a positive clone was selected and the insert thereof was confirmed not to contain any unexpected mutations. Thereafter, the plasmid of the selected clone was prepared and digested with SmaI. A DNA fragment 415 base pairs long was separated with agarose gel electrophoresis and then extracted for use in the subsequent ligation reaction.

2. Preparation of PUR DNA Fragments

A puromycin resistance gene (PUR) was amplified by PCR using a vector pPUR (BD Biosciences Clontech, Palo Alto, Calif., USA) which contains such gene, as a template. As a sense primer, an oligonucleotide comprising a SmaI site to be added at the 5′ end thereof was used. Also, as an antisense primer, an oligonucleotide comprising SmaI plus NaeI sites to be added at the 5′ end thereof was used. Further, in order to destroy the SmaI site in the vicinity of the 5′ end of PUR, mutation was introduced into the sense primer. Specifically, the sense primer #874′ (5′-tcc ccc ggg aag ctt acc atg acc gag tac aag ccc acg gtg cgc ctc gcc acc cgc gac gac gtc ccc agg-3′) (SEQ ID NO: 7) and the antisense primer #892 (5′-taa ccc ggg ccg gcg tca ggc acc ggg ctt g-3′) (SEQ ID NO: 8) were used to specifically amplify PUR with thermostable polymerase KOD-plus-.

The thus obtained DNA fragment was cloned into a pCR-Blunt I-TOPO vector using a Zero Blunt TOPO PCR Cloning Kit (Invitrogen, Carlsbad, Calif., USA). Then, a positive clone was selected and the insert thereof was confirmed not to contain unexpected mutation. Thereafter, the plasmid of the selected clone was prepared and digested with SmaI. A DNA fragment 621 base pairs long was separated with agarose gel electrophoresis and then extracted for use in the subsequent ligation reaction.

3. Insertion into SmaI Site of pcDNA3.1(+)

A pcDNA3.1(+) vector (Invitrogen, Carlsbad, Calif., USA) was digested with SmaI. Then, a linear DNA fragment 5428 base pairs long was separated with agarose gel electrophoresis and then extracted. Further, the DNA fragment was subjected to dephosphorilation) using calf intestinal alkaline phosphatase (CIAP) for use in the subsequent ligation reaction.

The DNA fragments of PUR and BSD as prepared above were ligated into the above SmaI-digested pcDNA3.1(+) vector using a Ligation Convenience Kit, respectively (FIG. 1 (B)). A positive clone was screened by direct PCR using primers #893 (5′-gtg tgg tgg tta cgc gca-3′) (SEQ ID NO: 9) and #894 (5′-cac aac tag aat gca gtg a-3′) (SEQ ID NO: 10), which had been designed to be located before and after the cleavage site. Further, the orientation of the insert was determined by PCR using these primers in combination with the primers used for preparations of the insert DNA fragments (#872, #891, #874′, and #892). The DNA sequences of the ligated sites of the vector sequences and the inserts were confirmed in the finally obtained clones.

4. Removal of Neo with NaeI Treatment

The clones as obtained above were digested with NaeI to produce DNA fragments of 3804 bp, 1062 bp, 694 bp, and 283 bp for BSD, and DNA fragments of 3804, 1268, 694, and 283 bp for PLUR These fragments correspond to a fragment derived from a vector backbone (pcDNA3.1(+)), a fragment derived from the BSD or PUR resistance gene, and fragments derived from the anterior half and the posterior half of the neomycin resistance gene, respectively (FIG. 1 (C)). Thus, the fragment derived from the vector background and the fragment derived from the BSD or PUR resistance gene were separated with agarose gel electrophoresis, extracted, and subjected to ligation reaction to remove the neomycin resistance gene therefrom (FIG. 1 (D)).

Upon selection of a clone, direct PCR was carried out to select a positive clone having a correctly oriented insert DNA using #893 and #894 as well as the primers used for preparations of the insert DNA fragments (#872, #891, #874′, #892). The DNA sequences of the ligated sites of the vector sequences and the inserts were confirmed in the thus finally obtained clones, and thus, pcDNA3.1(+)-PUR (SEQ ID NO: 2) and pcDNA3.1(+)-BSD (SEQ ID NO: 1) were obtained.

### Example 2

### Drug Resistance Test

pcDNA3.1(+), and pcDNA3.1(+)-PUR and pcDNA3.1(+)-BSD that had been newly constructed in the present invention were transfected into Chinese hamster ovary cells (CHO cells) using FuGENE6 (Roche Diagnostics Ltd., Basel, Switzerland), respectively, and then, surviving cells were selected in the presence of neomycin (I mg/ml), puromycin (10 microgram/ml), or blasticidin S (5 microgram/ml).

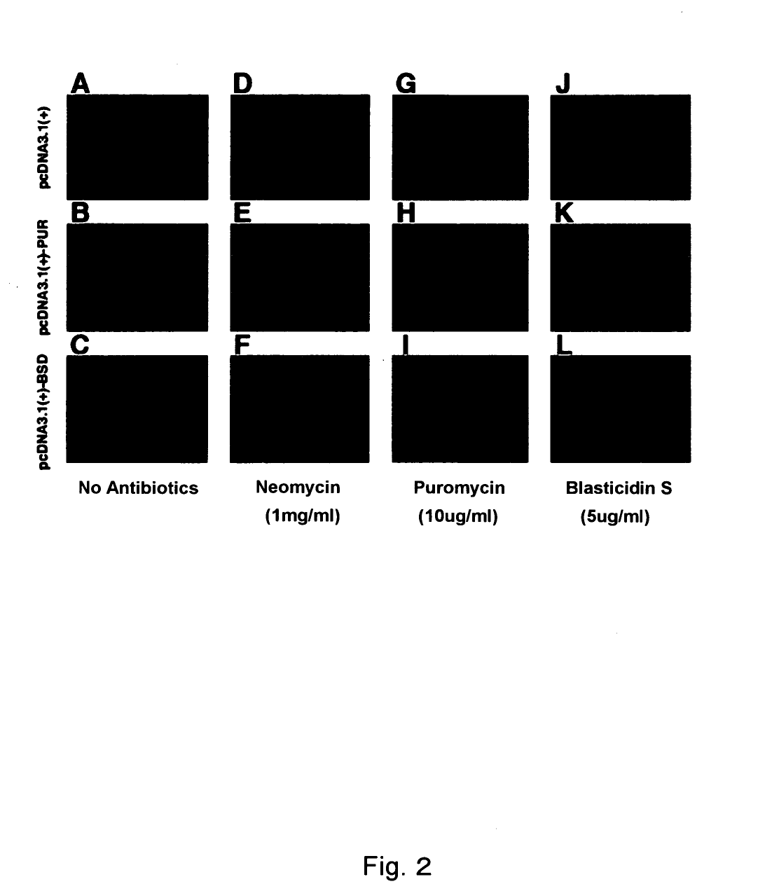

Each of the obtained drug resistance cells was seeded into 6 well plate containing antibiotic-free medium (alpha-MEM, 10% FCS) at 2.0×10⁵ cells per well. 7 hours later; the medium in each well was exchanged with a medium comprising neomycin (1 mg/ml), puromycin (10 micro-gram/ml), or blasticidin S (5 micro-gram/ml). Then, 3 days later, medium exchange was carried out so as to remove floating dead cells. Further, 3 days later, washing was carried out in a similar manner. Then photographs of the obtained cells were taken (FIG. 2).

Based on the photographs, sufficient cell proliferations were observed in the antibiotic-free medium for all tested cells (FIG. 2, panels A, B, and C). Due to the neomycin resistance gene contained in pcDNA3.1(+), the cells into which pcDNA3.1(+) had been transfected also survived and proliferated in the presence of neomycin (G418) (FIG. 2, panel D). However, such cells died in the presence of puromycin or blasticidin S (the panels G and 3).

In the case of the novel vectors pcDNA3.1(+)-PUR and pcDNA3.1(+)-BSD from which the neomycin resistance gene had been removed, the cells into which these vectors had been transfected, respectively, were not resistant against neomycin, and thus they died in the presence of neomycin (G418) (FIG. 2, panels E and F). Meanwhile, due to the puromycin resistance gene and the blasticidin S resistance gene respectively contained in these vectors, the cells into which these vectors had been transfected survived and proliferated in the presence of puromycin (FIG. 2, panel H) and in the presence of blasticidin S (FIG. 2, panel L), respectively.

What is claimed is:

1. An animal cell expression vector comprising a multicloning site and a drug resistance marker gene, the expression of which is controlled by SV40 ori and SV40 pA;
wherein the drug resistance marker gene is a puromycin resistance gene or a blasticidin S resistance gene.

2. The animal cell expression vector according to claim 1, wherein the multicloning site contains restriction enzyme recognition sites that are identical to those contained in a multicloning site of either pcDNA3.1(+) or pcDNA3.1(−), and the restriction enzyme recognition sites are located in the same order as those of pcDNA3.1(+) or pcDNA3.1(−).

3. A DNA fragment comprising a puromycin resistance gene in which SmaI recognition sites are added to both 3′ and 5′ ends of the puromycin resistance gene and an NaeI recognition site is added upstream of the SmaI recognition site on the 3′ end.

4. The DNA fragment according to claim 3, comprising the nucleotide sequence as set forth in SEQ ID NO: 4.

5. A DNA fragment comprising a blasticidin S resistance gene in which SmaI recognition sites are added to both 3′ and 5′ ends of the blasticidin S resistance gene and an NaeI recognition site is added upstream of the SmaI recognition site on the 3′ end.

6. The DNA fragment according to claim 5, comprising the nucleotide sequence as set forth in SEQ ID NO: 3.

7. A plasmid vector comprising the nucleotide sequence as set forth in SEQ ID NO: 1.

8. A plasmid vector comprising the nucleotide sequence as set forth in SEQ ID NO: 2.


SEQUENCE LISTING (10 sequences)

SEQ ID NO: 1 - DNA, 4866 bp, Artificial Sequence
  Expression vector (pcDNA3.1(+)-BSD)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggtcaatatgcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagcgatagtgaaggacagtgatggacagccgacggcagttgggattcgtgaattgctgccctctggttatgtgtgggagggctaagcaccagccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 5072 bp, Artificial Sequence
  Expression vector (pcDNA3.1(+)-PUR)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggaagcttaccatgaccgagtacaagcccacggtgcgcctcgccacccgcgacgacgtccccagggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 427 bp, Artificial Sequence
  DNA fragment for inserting  blasticidin S resistance gene
tcccccgggtcaatatgcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagcgatagtgaaggacagtgatggacagccgacggcagttgggattcgtgaattgctgccctctggttatgtgtgggagggctaagcaccagccggcccgggtta

SEQ ID NO: 4 - DNA, 633 bp, Artificial Sequence
  DNA fragment for inserting puromicin resistance gene
tcccccgggaagcttaccatgaccgagtacaagcccacggtgcgcctcgccacccgcgacgacgtccccagggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgccggcccgggtta

SEQ ID NO: 5 - DNA, 28 bp, Artificial Sequence
  chemically synthesized primer
tcccccgggtcaatatgcctttgtctca

SEQ ID NO: 6 - DNA, 31 bp, Artificial Sequence
  chemically synthesized primer
taacccgggccggctggtgcttagccctccc

SEQ ID NO: 7 - DNA, 72 bp, Artificial Sequence
  chemically synthesized primer
tcccccgggaagcttaccatgaccgagtacaagcccacggtgcgcctcgccacccgcgacgacgtccccagg

SEQ ID NO: 8 - DNA, 31 bp, Artificial Sequence
  chemically synthesized primer
taacccgggccggcgtcaggcaccgggcttg

SEQ ID NO: 9 - DNA, 18 bp, Artificial Sequence
  chemically synthesized primer
gtgtggtggttacgcgca

SEQ ID NO: 10 - DNA, 19 bp, Artificial Sequence
  chemically synthesized primer
cacaactagaatgcagtga